(19) United States
(12) Patent Application Publication
(10) Pub. No.: US 2018/0303493 A1
(43) Pub. Date: Oct. 25, 2018

(54) SURGICAL POWER TOOL

(71) Applicants: Robert CHAPOLINI, Baltimore, MD (US); BioAccess, Inc., Baltimore, MD (US)

(72) Inventor: Robert Chapolini, Baltimore, MD (US)

(21) Appl. No.: 15/765,472

(22) PCT Filed: Oct. 6, 2016
(86) PCT No.: PCT/US2016/055842

§ 371 Date: Apr. 2, 2018

Related U.S. Application Data
(60) Provisional application No. 62/238,590, filed on Oct. 7, 2015.

(51) Int. Cl. A61B 17/16; H01M 10/44; H01M 10/46

(52) U.S. Cl. CPC A61B 17/1622; A61B 2017/00734; H01M 10/46; H01M 10/44

(57) ABSTRACT

The present invention is a cordless, power tool having a primary battery that is rechargeable by a secondary battery, wherein the primary battery and/or motor can be separated from the tool prior to disposing the remainder of the tool, including a secondary battery.

### I. FIELD OF THE INVENTION

This invention relates to surgical power tools powered by using a novel combination of a rechargeable, reusable battery pack (lithium battery) with a disposable, non-rechargeable battery pack (non-lithium, e.g., alkaline).

### II. BACKGROUND OF THE INVENTION

State of the art cordless power tools are powered by rechargeable battery packs which must be charged and sterilized before each procedure. A minimum of two charged, sterilized battery packs are provided for each operative case that requires some type of surgical power tool. The charging of battery packs is typically performed in a prep room in the operating room area. The power tool, minus the battery pack, is generally prepared by the sterile supply department of the hospital, which is in a separate location. Processing of the power tool must be performed between each use, and it typically takes four to six hours between the time the tool leaves the operating room to the time it is returned, clean and sterilized.

Cordless reusable power tools do have significant disadvantages which are shared by all reusable power tools. They all need to be carefully cleaned before they are re-sterilized which takes time and hospital resources. These tools also require frequent maintenance from normal wear and, more significantly, from the deleterious effects of the high temperature, high pressure autoclave method of sterilization. Finally, surgical power tools which are processed in the hospital frequently have pyrogens on their surfaces. These pyrogens can cause complications for patients who are subsequently exposed to them.

One type of single-use power tool uses an alkaline (non-rechargeable) battery pack and a reusable motor unit that is inserted into the sterile housing at the beginning of the procedure. The alkaline battery pack is either integral to the sterile housing or attachable to the sterile housing. In either case, the alkaline battery pack is provided sterile to the user. At the end of the surgical procedure, the motor unit is removed from the housing; the entire tool, including the battery pack, is then disposed of using normal contaminated hospital waste methods. This type of single-use power tool requires no recharging of batteries and no cleaning or processing of any components. This configuration also has the greatest potential to reduce the costs of single-use surgical power tools since the motor is not disposed of as is the case of the previously described configurations.

However, this type of surgical power tool has one major limitation—power. More specifically, it is limited by the amount of input power that is available from alkaline batteries. Higher power, non-rechargeable lithium batteries are available and single-use power drills that incorporated this technology are currently available, but these cells cannot be disposed of with normal hospital waste. They must be recovered, electrically discharged, and then placed in controlled landfills. There are restrictions on the transport of these non-rechargeable lithium batteries. This recovery requirement reduces the convenience of this type of single-use power tool and adds additional back end costs to the user.

The actual time that a surgical power tool is used is extremely short, ranging from less than a minute to at most, several minutes. The total time, where the power tool is on the sterile field, but sitting in an idle condition is estimated at between 135 minutes for an orthopedic procedure to as much as 290 minutes in a cardiac surgery procedure. This extensive idle time is a unique condition, and presents a heretofore unrecognized opportunity to provide a battery operated disposable tool having sufficient power for virtually all applications and procedures.

The full benefits of single-use surgical power tools are not being realized in the presently available product offerings because of limitations of power and the failure to completely remove all of the processing steps that are associated with the use of conventional reusable power tools.

Also, single-use medical power tools with pre-charged capacitors or high energy batteries (e.g., lithium) constitute a potential shipping hazard due to an uncontained fire (thermal runaway). This makes routine shipping of new, sterile medical power tools with these high energy sources difficult and expensive.

The present invention seeks to overcome these limitations.

### III. SUMMARY OF THE INVENTION

This invention provides an electrical power source for single-use, cordless power tools which are designed for use in a sterile environment (e.g., in surgical procedures or in an operating room or theater). Examples of surgical power tools include, but are not limited to, rotary drills, reamers, staplers, and saws. The invention overcomes limitations of current single-use surgical power tools by using a novel combination of a rechargeable, reusable battery with a disposable, non-rechargeable battery. The non-rechargeable battery pack acts to 1) charge the rechargeable battery during idle conditions of the tool; and 2) augment the rechargeable battery during use of the power tool by providing a parallel input of electrical energy to the tool. Examples of non-rechargeable batteries include alkaline; examples of rechargeable packs include lithium and capacitors.

Further, the battery assembly of the present invention develops high power, single-use surgical tools. In addition, the battery configuration is adapted to eliminate the need for processing before use and recovery of batteries that cannot be safely disposed of using conventional medical waste disposal methods.

The battery pack/motor assembly or configuration contributes to another element of the invention—the single-use surgical power tool which is designed to be packaged sterile from the manufacturer and is disposed of after it is first used. The non-rechargeable second battery pack may be an integral part of the sterile surgical power tool, or it may be contained in a separate enclosure which can be attached or removed from the surgical power tool. The non-rechargeable second battery pack is packaged sterile from the manufacturer and is discarded after a single use.

In a sterile environment, there is an unmet need for a tool that provides disparate or inconsistent characteristics: high power, sterile, and disposable; an easily disposable cordless tool containing a lithium battery; a cordless tool that includes both sterile components and non-sterile components for use in a sterile field; a cordless tool having sufficient power in combination with a re-useable motor, where the ability to separate the motor from the tool does not infect the sterile field. There is also an unmet need for providing a high power, cordless power tool, that is sterile and pyrogen-free right out of its package.

Also, there is a need to provide a power tool in which any portion of the tool that becomes unsterile, e.g., comes into contact with a patient, can be safely disposed using conventional medical waste treatment methods. Power tools of the present invention include such a disposable portion.

Some embodiments of the invention also meet an unmet need: a disposable power tool having high power, a dual or multi-component battery system, uncorded, having no disposability issues, and no external battery charging.

With the following description of the drawings, the apparatus should become evident to a person of ordinary skill in the art.

### IV. BRIEF DESCRIPTION OF THE FIGURES

FIG. 1 illustrates an embodiment of the invention in which the second power source recharges the first power source.

FIG. 2 illustrates an embodiment of the invention in which the first power source is in a fully charged state and the power tool is in an idle condition.

FIG. 3 illustrates an embodiment of the invention in which the first power source and the second power source operate the motor.

FIG. 4 illustrates an embodiment of the invention in which the first power source operates the motor.

FIG. 5 shows an embodiment of the invention in which the housing or disposable portion is a drill.

FIG. 6 is a cross-section of an embodiment of the invention, showing an example of a configuration of first power source, second power source, and motor.

FIG. 7 is a bottom view section of the drill of FIG. 5, showing an example of a configuration of a motor assembly in the housing.

FIG. 8 is a section view of the drill of FIG. 5, showing an example of a second power source configuration.

FIG. 9 is an example of a motor assembly of one embodiment of the invention.

FIG. 10 is an exploded side view of the connection between the motor assembly and disposable portion of the tool.

### V. DETAILED DESCRIPTION OF THE INVENTION

The present invention is an uncorded power tool having a disposable portion and a non-disposable portion, wherein the non-disposable portion contains at least one first power source configured or shaped to be disposed within a housing; and wherein the disposable portion contains at least one second power source disposed within the housing. In preferred embodiments of the invention, the non-disposable portion also includes at least one motor. In the most preferred embodiments of the invention, the non-disposable portion is a motor assembly comprising at least one motor and at least one first power source.

In preferred embodiments of the invention, the first power source is rechargeable and/or not disposable, e.g., a lithium battery or a capacitor. In some of these embodiments, the first power source is re-usable.

The disposable portion comprises a second power source for charging or re-charging the first power source. In this embodiment of the invention, the non-disposable, non-sterile portion is configured to be positioned inside a sleeve of the disposable portion, thereby making the power tool sterile for its intended use. In these embodiments of the invention, both the non-disposable portion and the disposable portion are required to make the power tool functional.

In some embodiments of the invention, the second power source is disposable and/or has a long shelf life (i.e., does not tend to self-discharge), e.g., an alkaline battery. Typically, a disposable power source is not re-usable and/or is not re-chargeable.

In accordance with the present invention, the surgical power tool, when ready for operation, is sterile and pyrogen free. A power tool of the present invention also does not require re-charging batteries from an external source.

In preferred embodiments of the invention, the power tool is intended for use in a sterile environment. In some of these embodiments, the power tool is a surgical power tool.

The present invention is also a battery system for powering a power tool, wherein the system comprises a first power source and a second power source, wherein the first power source is re-chargeable and not disposable; and the second power source is configured to charge the first power source, said second power source being separable from said first power source and disposable.

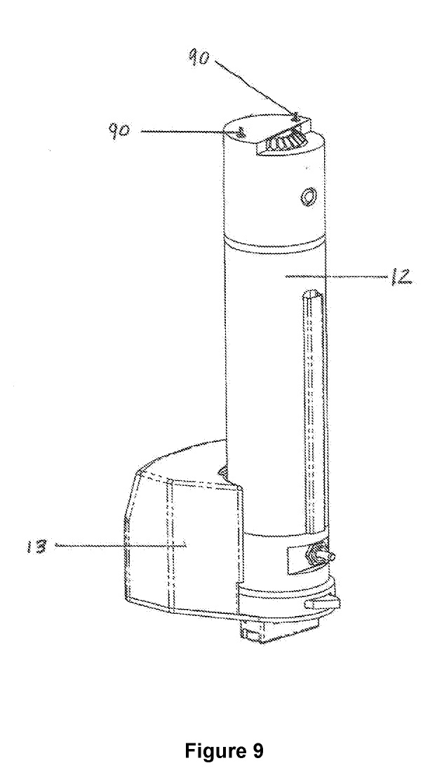

An embodiment of the invention is a power tool having a disposable portion and a non-disposable portion. The non-disposable portion comprises a first power source and a motor, said first power source and motor being non-sterile. An exemplary non-disposable portion is shown in FIG. 9. The disposable portion comprises a second power source for charging or re-charging the first power source. In this embodiment of the invention, the non-disposable non-sterile portion is configured to be positioned inside a sleeve of the disposable portion, thereby making the power tool sterile for its intended use. In these embodiments of the invention, both the non-disposable portion and the disposable portion are required to make the power tool functional.

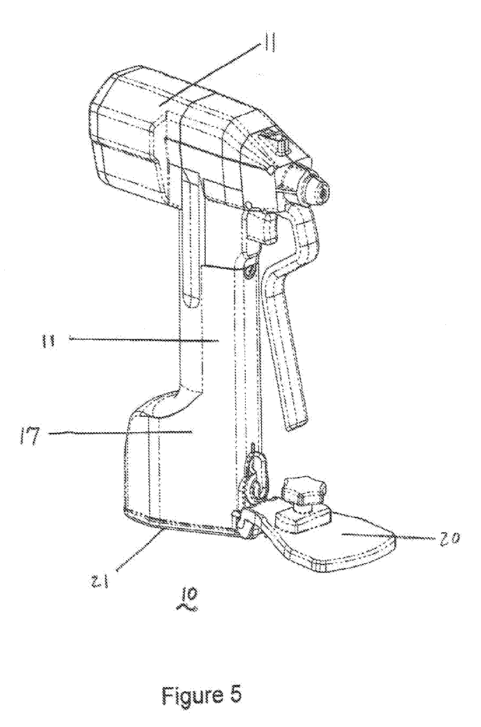

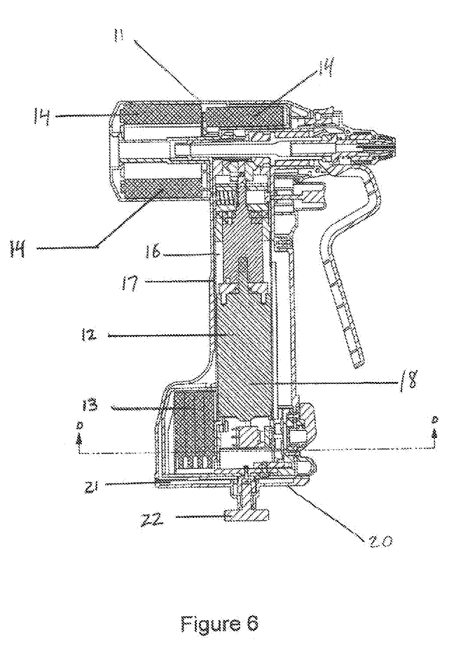

In the exemplary embodiment shown in FIGS. 5 and 6, everything pictured in FIG. 5 is disposable; and in FIG. 6, everything is disposable except first power source 13, motor 12, and motor assembly 18.

The present invention involves a disposable surgical power tool comprising: a housing, suitable for use in a surgical environment; a motor configured for placement in the housing, said motor being optionally removable; a first power source positioned in the housing, wherein said first power source is rechargeable, and wherein the first power source is optionally removable with or without the motor; a second power source positioned in the housing, in aseptic communication with the first power source; a control circuit for controlling electrical current between various components of the tool, e.g., between the motor, first power source, and the second power source; wherein, in a first operating function, the first power source is configured to operate the motor; and in a second operating function, the second power source recharges the first power source; and in a third operating function, the first power source and the second power source operate a motor.

In some embodiments of the invention, the tool and method include an optional fourth operating function, in which the second power source operates the motor and the second power source recharges the first power source.

The present invention also involves a power source for a disposable tool, wherein the power source comprises a rechargeable and removable battery; and a non-rechargeable and disposable battery. In some embodiments of the inventions the removable battery may be removed independently of the motor, and in other embodiments, the removable battery and the motor may be removed together.

The invention also involves a method for using a power tool in a sterile environment, such as during surgery or the like, where, typically, the desire for sterility and/or pyrogen-free conditions is important. In preferred embodiments of the invention, the method involves using a sterile tool having one or more internal components that are non-sterile and/or non-sterilizable. In these embodiments of the invention, the non-sterile component of the tool refers to the motor, the first power source, and combinations thereof.

In the most preferred embodiments of the invention, the first power source is attached to a motor, thereby forming a non-disposable motor assembly. An exemplary configuration of a motor assembly 18 is shown in FIG. 9.

The present invention also includes a power tool comprising a non-disposable battery pack assembly. In some embodiments of the invention, the power tool is a medical or surgical power tool.

In other embodiments, the method involves using a sterile tool having a disposable portion and a non-disposable portion. In these embodiments of the invention, the disposable portion is typically single-use or of such limited use that the portion can be thrown away or discarded. In these embodiments of the invention, the disposable portion includes the second power source.

The surgical power tool is always sterile (at least at the beginning of the operation), the primary battery is never sterile, and the secondary battery is either an integral part of the sterile power tool or is a removable/replaceable sterile module.

Exemplary power tools of the present invention are shown in FIGS. 5-8. In FIG. 5, a disposable power tool is generally denoted as 10. The illustrated power tool is a cordless (uncorded) surgical drill. The power tool 10 comprises a housing 11. Housing 11 comprises a sleeve 17. Sleeve 17 comprises an internal portion 16 or cavity configured to receive a motor 12, a first or primary power source 13, or a motor assembly 18. This embodiment also shows the cover or cap 20 in an open position, said cover being configured to close or cover port or opening 21 in sleeve 11.

FIG. 6 shows one or many examples of possible configurations in accordance with the present invention. In this example, motor 12 is in communication with, or attached to, a first or primary power source 13. Second or secondary power source 14 is positioned in the housing 11 and is electrically connected to the motor 12 and the first power source 13. FIG. 6 also illustrates the operable condition of the power tool in its sterile and pyrogen-free state, e.g., with motor 12 enclosed within the housing 11 and the cap 20 in a closed position. In this embodiment of the invention, cap 20 covers port 21, said port being configured to receive the motor, the primary battery, or both. FIG. 6 also shows a screw 22 for closing the cap 20 in a sealing condition over sleeve 17.

FIG. 6 is also an exemplary illustration of a fully functional power tool 10, having a non-disposable portion (motor 12 and first power source 13; or motor assembly 18) and a disposable portion, wherein the non-disposable portion is sealed within the disposable portion.

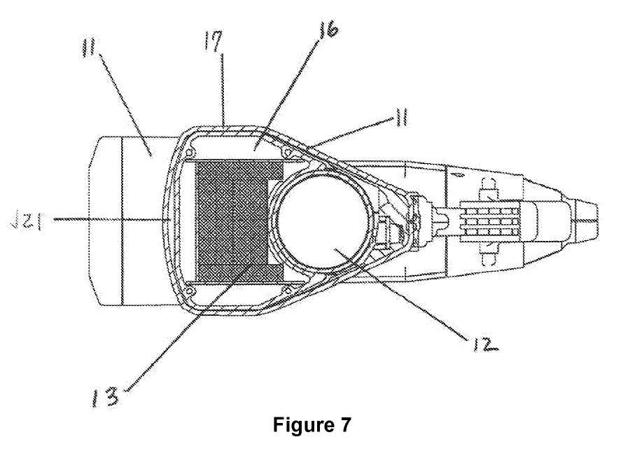

FIG. 7 is a bottom view of a power tool 10 (without cover 20), and shows another view of the motor 12 and first power source 13 positioned in the cavity 16 of sleeve 17

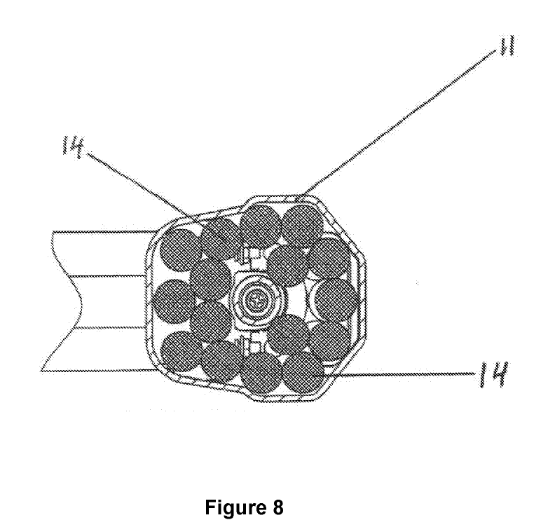

FIG. 8 shows the portion of housing 11 that is configured to position the second power source 14. In the illustrated embodiment, the second power source, e.g., alkaline batteries, are configured into a pack.

FIG. 9 illustrates an embodiment of the invention in which the motor 12 and first power source 13 are configured into a motor assembly 18. FIG. 9 also shows motor connector 90 or contacts that permit connection between the motor and the second power source. In preferred embodiments of the invention, these contacts are spring-loaded.

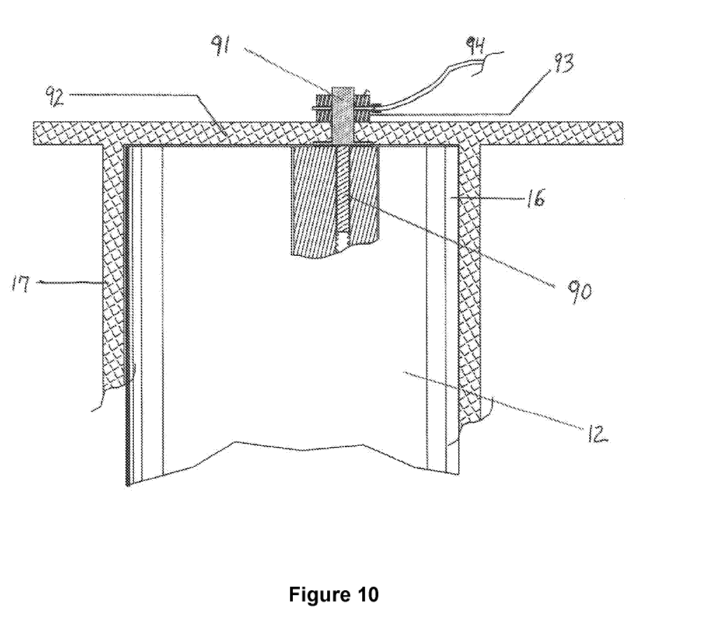

FIG. 10 also illustrates an embodiment of the invention showing the connection between the motor 12 and the second power source (not shown). This figure also illustrates an exemplary embodiment of the connection between the non-disposable portion of the tool and the disposable portion. This figure also illustrates an exemplary embodiment of a configuration in which the non-sterile portion (e.g., motor 12) is aseptically connected to a sterile portion of the tool.

In the illustrated embodiment motor 12 is positioned in a cavity 16 of sleeve 17, and in the cut-away section of the illustration, a portion of the motor (e.g., spring-loaded contact(s) 90) touch or contact stud 91 positioned through an internal wall 92 of sleeve 17. Contact stud 91 is preferably sealed within wall 92, marking the boundary between the sterile portion of the tool and the non-sterile motor. Contact stud 91 may be further secured to or within the wall with a nut 93 or the like. The connection between the stud and the nut may further include a gasket or sealant material (not shown), another element intended to provide or maintain an aseptic condition within the tool between a sterile portion of the tool and the non-sterile motor. FIG. 10 also shows wire 94 attached to stud 91 for connecting the motor to the second power source (not shown) or other elements of the electrical system within the power tool.

First Power Source

The first power source is at least one battery or capacitor capable of providing high power and is rechargeable. In preferred embodiments of the invention, the first power source is a lithium battery. Typically, these kinds of batteries cannot legally be disposed or discarded in normal hospital waste. These kinds of batteries must be recovered (e.g., separated from the tool they are powering), electrically discharged, and then placed in a controlled landfill. At the present time, batteries such as lithium batteries are very limited in their ability to transport, in part due to the danger of explosion and/or fire.

Two or more primary power sources may be linked together, e.g., as a pack, to provide power to the motor. Two or more primary batteries may be configured into a primary battery pack.

The primary battery typically retains its charge for a shorter time compared to the secondary battery. The primary power source also typically is a low impedance device that allows it to deliver large amounts of power and/or larger amounts of power relative to the second power source. Therefore, a sterile power tool which includes a secondary battery (either integral or replaceable) has a very long shelf life compared to a power tool with an integral or replaceable sterile primary battery.

In accordance with a preferred embodiment of the invention, configuring the first power source and the motor into a motor assembly within the housing is advantageous because neither element is typically sterilized or sterilizable. This is usually accurate for the motor also, although some motors may be sterilizable, but doing so may degrade or limit the longevity of the motor.

Second Power Source

The second power source, or secondary battery, is at least one battery suitable for use to recharge the primary battery. In preferred embodiments of the invention, the second power source is an alkaline battery, a nickel/cadmium battery, or combinations thereof. In the most preferred embodiments of the invention, the secondary battery is disposable.

In accordance with the present invention, the primary purpose of the secondary power source is to charge and/or recharge the primary power source. In some embodiments of the invention, the secondary battery may also provide power to the motor, typically by supplementing or adding to the power provided by the first power source.

In accordance with the present invention, primary characteristics of the secondary battery are that it is disposable and it has a long shelf life.

Two or more secondary batteries may be linked together, e.g., as a pack, to provide power to the primary battery and/or to the motor. Two or more secondary batteries may be configured into a secondary battery pack.

Uses/Methods

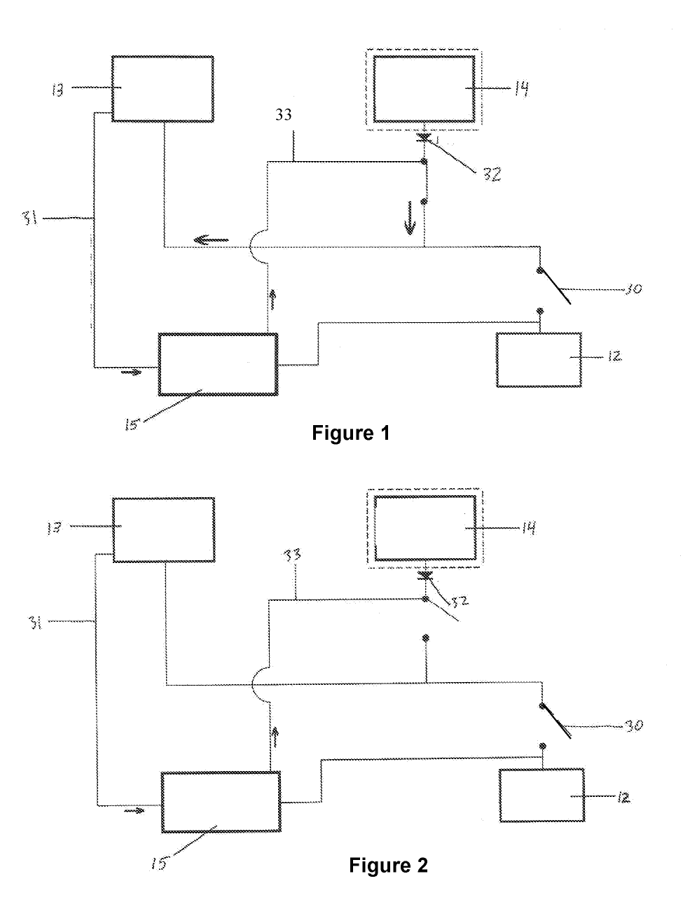

FIGS. 1-4 illustrate an embodiment of the invention in use. The first operating function is when the primary battery operates the motor, illustrated in FIG. 4.

The second operating function is when the second power source charges or recharges the first power source, illustrated in FIG. 1.

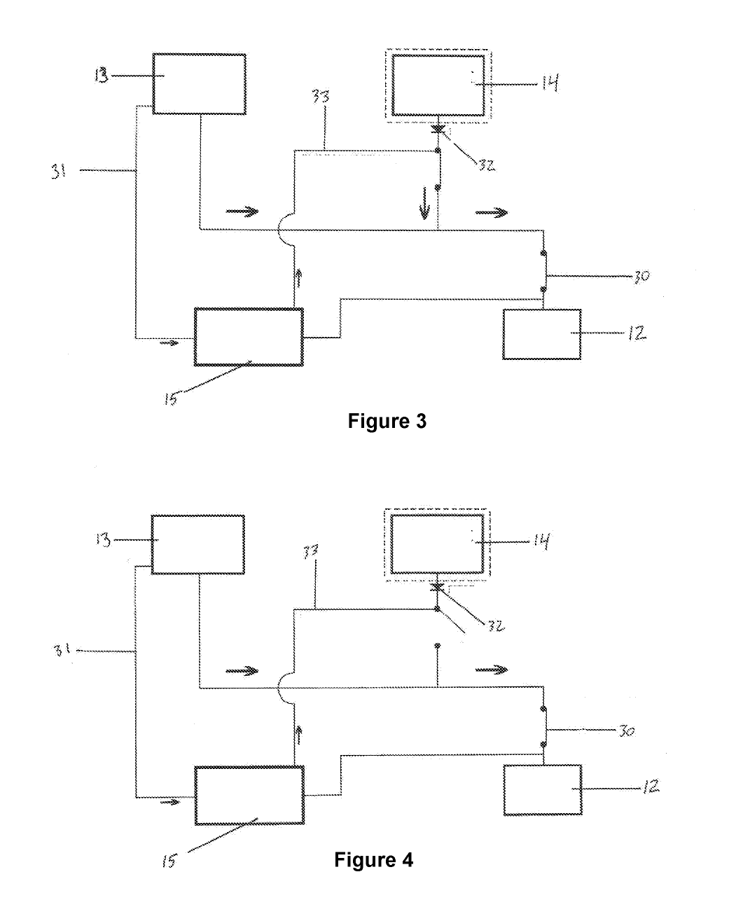

The third operating function is when both the first power source and the second power source operate the motor, illustrated in FIG. 3. If the potential of the secondary power source is higher than the potential of the primary power source, the secondary will power the motor and charge the primary simultaneously.

In some embodiments of the invention, the secondary battery charges or recharges the primary battery prior to the initial use of the tool, e.g., to assure that the primary battery is fully charged prior to use. The secondary battery may also be used to recharge the primary battery, e.g., after an initial use of the tool when the primary battery has been partly or wholly discharged. FIG. 2 illustrates the power tool in an idle condition and the primary power source in a fully charged condition. This is an exemplary configuration of the tool prior to use, or ready for use after a period of non-use (e.g., for a period sufficient for the second power source to re-charge the first power source).

As noted above, a tool, power source, or system of the present invention does not need to be charged or re-charged using an external power source.

In accordance with the present invention, all of the functions noted above may be performed using elements known to those with skill in the art, e.g., electrical connectors, switches, sensors, controllers, and the like.

Once the rechargeable first and the non-rechargeable second battery packs are physically attached to the surgical power tool and also when the power tool is in an idle condition, electrical current can flow from the non-rechargeable second battery pack to the rechargeable first battery pack if the rechargeable first battery pack is found to be below a fully charged condition. An electronic regulating circuit can monitor the condition of the rechargeable first battery pack and can control the rate of charging of the rechargeable first battery pack; this monitor, or another monitor, can terminate charging the rechargeable first battery pack when it is at a fully charged condition. The electronic control circuit, or another circuit, may prevent the flow of electrical current from the rechargeable first battery pack to the non-rechargeable second battery pack.

At any time when the surgical power tool is operated by the user, power to the motor can be supplied by the primary battery alone or, if the voltage of the primary battery falls below a critical value, the electrical regulating circuit can cause both the primary and secondary battery packs to be connected together, in parallel. When the surgical power tool returns to an idle condition, the first and second battery packs are connected so that charging of the rechargeable first battery pack by the non-rechargeable second battery pack may resume, if the electronic regulating circuit determines that the rechargeable first battery pack is not at a fully charged condition.

In some embodiments of the invention, the power tool may be used intermittently. Typically, the primary battery may be discharged or partially discharged. The present invention is adapted and configured to address this situation by providing a secondary battery that can recharge the primary battery. A tool of the present invention does not need to be attached or connected to an external power source to recharge the primary battery.

Once use of the tool is completed, (e.g., the operation or surgery is finished), the now non-sterile power tool must be disposed. As noted above, the primary battery must be carefully and separately disposed or recycled because there is a fire hazard, such as with high energy lithium batteries. This might also apply to a charged capacitor or the like. These components of the tool need to be separated from the tool, so in preferred embodiments of the invention, the tool is configured so that the primary battery and any other dangerous components may be separated from the tool. In some embodiments of the invention, the primary power source and/or the motor may be used multiple times, and is preferably re-used over and over again. In these embodiments, the primary power source and/or the motor can be placed in a new (sterile) disposable portion of the power tool.

Likewise, the motor may be recycled or re-used. In accordance with some embodiments of the invention, the motor may be separated or removed from the tool.

The remainder of the tool, e.g., the housing and the secondary battery may be disposed using normal procedures, e.g., following typical medical waste disposal procedures.

Miscellaneous

FIGS. 1-4 also illustrate that a power tool of the present invention may also include other elements, elements that are known to those with skill in the art. These figures show a tool with

In accordance with some embodiments of the invention, one or more of the motor, the primary battery, and the secondary battery may not be sterile in itself, but the patient would be protected if the element was positioned within the housing of the tool.

As noted above, some embodiments of the invention include a sterile portion and an internal non-sterile and/or re-usable portion. When the cap is in its fully closed position, the housing fully encloses any non-sterile and/or re-usable components (e.g., the primary battery and/or the motor), thus maintaining the sterility of the power tool prior to use.

As noted in FIGS. 5 and 6, the tool may include a cap (20) for closing access to the internal portion of the housing. The cap may further include a screw 22 or the like for sealing the closed cap.

A power tool of the present invention may include a control circuit 15 (shown in FIG. 1). In some embodiments of the invention, the control circuit may monitor the charge of the primary battery and prevent overcharging. This control circuit, or another, may also monitor the position of a throttle sensor to determine which of one or more operating functions are desirable or necessary.

In some embodiments of the invention, a diode or the like prevents current from returning or entering the secondary battery.

In accordance with some embodiments of the invention, the power tool is disposable and a portion of the tool, e.g., the motor and/or the primary battery, must first be separated from the tool prior to disposal.

A preferred embodiment of the invention would have the rechargeable first battery pack contained within a reusable module which could contain other elements that are required to operate the sterile surgical power tool, such as electronics, gear trains and/or one or more motors. The reusable module containing the rechargeable first battery pack is insertable inside a compartment of the sterile surgical power tool, and which can be sealed within by a cover, thereby sealing the reusable module inside the sterile surgical power tool. The non-rechargeable second battery pack could be integral to the sterile surgical power tool or configured as a separate sterile module that is removably attached to the sterile surgical tool. At the end of the surgical procedure, the reusable module is removed from the now contaminated surgical power tool and retained for subsequent use. The contaminated surgical power tool and non-rechargeable second battery pack are disposed of using normal hospital contaminated waste procedures.

Another alternative embodiment of the invention includes a sleeve configured as two separate channels, one channel for the motor and one channel for the first power source; or a housing with two separate sleeves.

An alternate embodiment of the invention would have the rechargeable first battery pack, as a separate element, insertable inside a compartment of the sterile surgical power tool, and which can be sealed within by a cover, thereby sealing the rechargeable first battery pack inside the sterile surgical power tool. The non-rechargeable second battery pack could be integral to the sterile surgical power tool or configured as a separate sterile module that is removably attached to the sterile surgical tool. At the end of the surgical procedure, the rechargeable first battery pack is removed from the now contaminated surgical power tool and retained for subsequent use. The contaminated surgical power tool and non-rechargeable second battery pack are disposed of using normal hospital contaminated waste procedures.

In accordance with the present invention, the motor or first power source may be connected to the second power source through an aseptic connection. An exemplary embodiment of this feature is shown in FIG. 10, where the non-sterile motor comprises at least one connector configured to contact a contact stud positioned in a wall of the sleeve. In preferred embodiments of the invention, the contact stud or connector is sealed in the wall using any structure that seals the connections, thereby maintaining sterility on one side of the wall. In these embodiments of the invention, the sleeve wall separates the sterile portion of the tool from the non-sterile. The sleeve wall also demarcates the disposable portion of the tool from the non-disposable.

A tool of the present invention may also include one or more status indicators, such as LED lights, that provide condition indicators for the motor, the primary battery and/or the secondary battery.

One skilled in the art will recognize that the invention as described here may be reconfigured into different combinations, elements, and processes which are included within the scope of the invention.

### Definitions

The following definitions are used in reference to the invention:

(A) A first power source may be combined with at least one additional first power source; such a configuration used herein as a primary power pack or a primary battery pack. A secondary power source may be combined with at least one additional secondary power source; such a configuration used herein as a secondary power pack or a secondary battery pack.

(B) Recycling used power tools away from the point of use is an important feature of the invention. It would be unsafe to send a used power tool with high energy batteries (the primary battery) or even a charged capacitor away for reprocessing. Sending out a power tool with the low power secondary battery is not an issue.

While the invention has been described in some detail by way of illustration and example, it should be understood that the invention is susceptible to various modifications and alternative forms, and is not restricted to the specific embodiments set forth in the Examples. It should be understood that these specific embodiments are not intended to limit the invention but, on the contrary, the intention is to cover all modifications, equivalents, and alternatives falling within the spirit and scope of the invention.

1. A power tool suitable for use in a sterile environment, comprising:
A. a disposable portion comprising a hollow sleeve;
B. a re-usable motor configured for insertion within the sleeve;
C. a first power source, configured for electrical communication with said motor, said first power source being re-chargeable and re-usable; said first power source being configured for insertion is said sleeve;
D. A second power source configured for positioning within said disposable portion, said second power source being configured for charging or re-charging said first power source.

2. A power tool comprising:
a housing, suitable for use in a sterile environment;
a motor configured for placement in the housing;
a first power source positioned in the housing, wherein said first power source is rechargeable;
a second power source positioned in the housing, in communication with the first power source;
a control circuit between the motor, first power source, and the second power source;
wherein:
in a first operating function, the first power source is configured to operate the motor; and
in a second operating function, the second power source recharges the first power source; and
in a third operating function, the first power source and the second power source operate a motor.

3. The power tool of claim 1 wherein the motor and the first power source are non-sterile.

4. The power tool of claim 3 wherein the connection between the non-sterile motor and the second power source is an aseptic connection.

5. The power tool of claim 1 wherein the tool is a surgical tool.

6. The power tool of claim 2 further comprising a control circuit adapted and configured to select among and between one or more of the operating functions.

7. The power tool of claim 2 wherein the second power source powers the motor and charges the first power source.

8. A power source for a disposable tool comprising:
a first power source, wherein said first power source is rechargeable;
a second power source, in communication with the first power source, wherein said second power source is disposable;
wherein:
in a first operating function, the first power source is configured to operate a motor; and
in a second operating function, the second power source recharges the first power source; and
in a third operating function, the first power source and the second power source operate the motor

9. The power source of claim 8 further comprising a control circuit adapted and configured to select among and between one or more of the operating functions.

10. The power tool of claim 1 wherein the first power source and the motor are configured into an assembly.

11. The power tool of claim 1 wherein the power tool cannot be charged or re-charged from an external power source.

12. A method of using a power tool in a sterile environment comprising:
providing a power tool, said power tool comprising a disposable portion, wherein said disposable portion comprises a hollow sleeve a non-sterile motor configured to insertion in said sleeve; a rechargeable first power source; and a second power source disposed within the disposable portion and in communication with said first power source and the motor;
operating said power tool.

13. The method of claim 12 wherein said first power source is non-sterile and is configured to be inserted into said power tool while maintaining the sterility of the environment in which the power tool is used.

14. The method of claim 12 further comprising
removing said first power source and motor from said tool; and
disposing of the disposable portion of the tool.
